(19) United States
(12) Patent Application Publication
(10) Pub. No.: US 2013/0338184 A1
(43) Pub. Date: Dec. 19, 2013

(54) 2-AMINO-NAPHTHYRIDINE DERIVATIVES

(71) Applicant: Concert Pharmaceuticals Inc., Lexington, MA (US)

(72) Inventor: I. Robert Silverman, Arlington, MA (US)

(21) Appl. No.: 13/973,810

(22) Filed: Aug. 22, 2013

Related U.S. Application Data
(63) Continuation-in-part of application No. PCT/US2012/026536, filed on Feb. 24, 2012.
(60) Provisional application No. 61/446,733, filed on Feb. 25, 2011.

(51) Int. Cl. C07D 471/04

(52) U.S. Cl. CPC C07D 471/04
USPC 514/300; 546/122

(57) ABSTRACT

The invention in one embodiment is directed to a compound of Formula A:

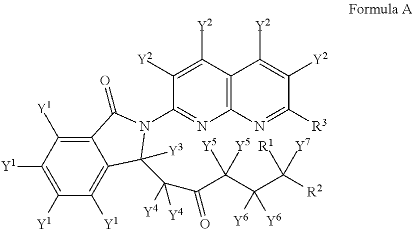

as defined herein, or a pharmaceutically acceptable salt thereof.

### CROSS-REFERENCE TO RELATED APPLICATIONS

This application is a continuation-in-part and claims priority under 35 U.S.C. §120 to PCT/US2012/026536, filed Feb. 24, 2012, which claims the benefit of priority to U.S. Provisional Application No. 61/446,733, filed Feb. 25, 2011, the contents of which are incorporated by reference herein in their entirety.

### BACKGROUND OF THE INVENTION

Many current medicines suffer from poor absorption, distribution, metabolism and/or excretion (ADME) properties that prevent their wider use or limit their use in certain indications. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment. A rapidly metabolized drug may also expose patients to undesirable toxic or reactive metabolites.

Another ADME limitation that affects many medicines is the formation of toxic or biologically reactive metabolites. As a result, some patients receiving the drug may experience toxicities, or the safe dosing of such drugs may be limited such that patients receive a suboptimal amount of the active agent. In certain cases, modifying dosing intervals or formulation approaches can help to reduce clinical adverse effects, but often the formation of such undesirable metabolites is intrinsic to the metabolism of the compound.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, the CYP2D6 inhibitor quinidine has been added to dextromethorphan for the purpose of reducing rapid CYP2D6 metabolism of dextromethorphan in a treatment of pseudobulbar affect. Quinidine, however, has unwanted side effects that greatly limit its use in potential combination therapy (see Wang, L et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug or to reduce the formation of undesirable metabolites by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

This invention relates to novel substituted isoindolones, their derivatives, and pharmaceutically acceptable salts thereof. This invention also provides compositions comprising a compound of this invention and the use of such compositions in methods of treating diseases and conditions that are beneficially treated by administering a GABA-A receptor modulator.

Pagoclone, also known as (+)-2-(7-chloro-1,8-naphthyridin-2-yl)-3-(5-methyl-2-oxohexyl)isoindolin-1-one, is a GABA-A receptor modulator that acts at the benzodiazepine site of the GABA-A receptor. Pagoclone is in Phase III clinical trials for persistent developmental stuttering (PDS). Despite the beneficial activities of pagoclone, there is a continuing need for new compounds that are GABA-A receptor modulators.

### DEFINITIONS

The term “treat” means decrease, suppress, attenuate, diminish, arrest, or stabilize the development or progression of a disease (e.g., a disease or disorder delineated herein).

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of pagoclone will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66:15; Gannes, L Z et al., Comp Biochem Physiol Mol Integr Physiol, 1998, 119:725. In a compound of this invention, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation) at each atom designated as deuterium in said compound.

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopologue” refers to a species the chemical structure of which differs from a specific compound of this invention only in the isotopic composition thereof.

The term “compound,” as used herein, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues will be less than 49.9% of the compound.

The invention also includes salts of the compounds disclosed herein.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds of the present invention (e.g., compounds of Formula A), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert”, “^{t}”, and “t-” each refer to tertiary. “US” refers to the United States of America.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

In one embodiment, the invention is directed to a compound of Formula A:

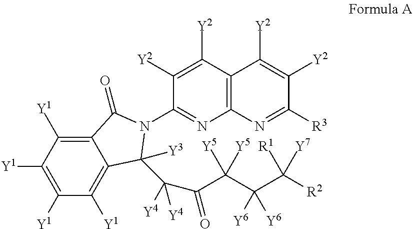

or a pharmaceutically acceptable salt thereof, wherein:

each Y¹ is the same and is hydrogen or deuterium;

each Y² is the same and is hydrogen or deuterium;

Y³ is hydrogen or deuterium;

each Y⁴ is the same and is hydrogen or deuterium;

each Y⁵ is the same and is hydrogen or deuterium;

each Y⁶ is the same and is hydrogen or deuterium;

Y⁷ is OR⁴, hydrogen or deuterium;

R¹ is CH₃ or CD₃;

R² is CH₃ or CD₃;

R³ is Cl, CH₃ or CD₃; and

R⁴ is hydrogen or P(O)(OR⁵)₂; wherein

each R⁵ is independently hydrogen or C₁-C₆ alkyl, provided that at least one R⁵ is C₁-C₆ alkyl;

provided that when each Y is hydrogen, at least one of R′, R² and R³ is CD₃.

In one embodiment, the compound of Formula A is a compound of Formula I:

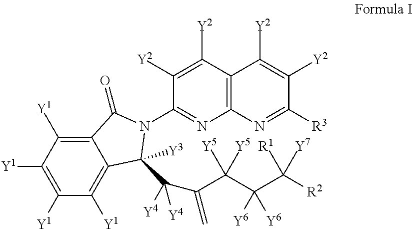

or a pharmaceutically acceptable salt thereof, wherein:

each Y¹ is the same and is hydrogen or deuterium;

each Y² is the same and is hydrogen or deuterium;

Y³ is hydrogen or deuterium;

each Y⁴ is the same and is hydrogen or deuterium;

each Y⁵ is the same and is hydrogen or deuterium;

each Y⁶ is the same and is hydrogen or deuterium;

Y⁷ is OR⁴, hydrogen or deuterium;

R¹ is CH₃ or CD₃;

R² is CH₃ or CD₃;

R³ is Cl, CH₃ or CD₃; and

R⁴ is hydrogen or P(O)(OR⁵)₂; wherein

each R⁵ is independently hydrogen or C₁-C₆ alkyl, provided that at least one R⁵ is C₁-C₆ alkyl;

provided that when each Y is hydrogen, at least one of R′, R² and R³ is CD₃.

In one embodiment, the compound of Formula I is a compound of the Formula Ia

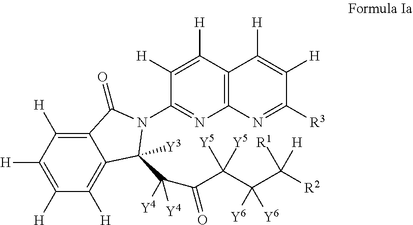

or a pharmaceutically acceptable salt thereof,
wherein:

Y³, Y⁴, Y⁵, Y⁶, R¹, R² and R³ are each defined as in formula I, and wherein R¹ and R² are the same.

In one embodiment, the compound of Formula I is a compound of the Formula Ib

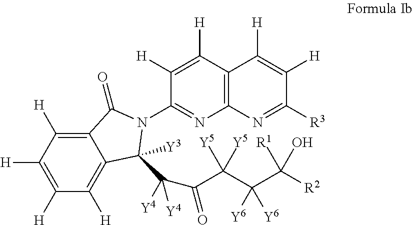

or a pharmaceutically acceptable salt thereof,
wherein:

Y³, Y⁴, Y⁵, Y⁶, R¹, R² and R³ are each defined as in formula I, and wherein R¹ and R² are the same.

In one embodiment, the compound of Formula I is a compound of the Formula Ic

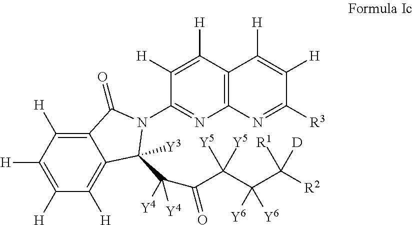

or a pharmaceutically acceptable salt thereof,
wherein

Y³, Y⁴, Y⁵, Y⁶, R¹, R² and R³ are each defined as in Formula I, and wherein R¹ and R² are the same.

In one embodiment of formula I or Ia or Ib or Ic, each Y⁶ is deuterium. In one aspect of this embodiment, R¹ and R² are each CD₃. In another aspect, R¹ and R² are each CH₃. In one aspect of this embodiment, R³ is Cl. In another aspect, R³ is CD₃. In one aspect of this embodiment, each Y⁵ is hydrogen. In another aspect, each Y⁵ is deuterium. In one aspect of this embodiment, each Y⁴ is hydrogen. In another aspect, each Y⁴ is deuterium.

In one embodiment of formula I or Ia or Ib or Ic, R¹ and R² are each CD₃. In one aspect of this embodiment each Y⁶ is hydrogen. In one aspect of this embodiment, R³ is Cl. In another aspect, R³ is CD₃. In one aspect of this embodiment, each Y⁵ is hydrogen. In another aspect, each Y⁵ is deuterium. In one aspect of this embodiment, each Y⁴ is hydrogen. In another aspect, each Y⁴ is deuterium.

In another set of embodiments, any atom not designated as deuterium in any of the embodiments set forth above is present at its natural isotopic abundance.

In one embodiment of the compound of Formula I, Y³, each Y¹, each Y², each Y⁴ and each Y⁵ are all hydrogen, R¹ and R² are the same, and the compound is selected from any one of the compounds set forth in Table 1a below:

TABLE 1a
Examples of Compounds of Formula I

| Compound | R¹ = R² | R³ | each Y⁶ | Y⁷ |
|---|---|---|---|---|
| 101 | CD₃ | Cl | D | OH |
| 102 | CD₃ | Cl | D | H |
| 103 | CD₃ | Cl | H | OH |
| 104 | CD₃ | Cl | H | H |
| 105 | CD₃ | CD₃ | D | OH |
| 106 | CD₃ | CD₃ | D | H |
| 107 | CD₃ | CD₃ | H | OH |
| 108 | CD₃ | CD₃ | H | H |
| 109 | CH₃ | Cl | D | OH |
| 110 | CH₃ | Cl | D | H |
| 111 | CH₃ | Cl | H | OH |
| 112 | CH₃ | CD₃ | D | OH |
| 113 | CH₃ | CD₃ | D | H |
| 114 | CH₃ | CD₃ | H | OH |
| 115 | CH₃ | CD₃ | H | H |

or a pharmaceutically acceptable salt thereof, wherein any atom not designated as deuterium is present at its natural isotopic abundance.

In one embodiment of the compound of Formula I, Y³, each Y¹, each Y², each Y⁴ and each Y⁵ are all hydrogen, R¹ and R² are the same, and the compound is selected from any one of the compounds set forth in Table 1b below:

TABLE 1b
Examples of Compounds of Formula I

| Compound | R¹ = R² | R³ | each Y⁶ | Y⁷ |
|---|---|---|---|---|
| 121 | CD₃ | Cl | D | D |
| 122 | CD₃ | Cl | H | D |
| 123 | CD₃ | CD₃ | D | D |
| 124 | CD₃ | CD₃ | H | D |
| 125 | CH₃ | Cl | D | D |
| 126 | CH₃ | Cl | H | D |
| 127 | CH₃ | CD₃ | D | D |
| 128 | CH₃ | CD₃ | H | D |

or a pharmaceutically acceptable salt thereof, wherein any atom not designated as deuterium is present at its natural isotopic abundance.

In one embodiment of the compound of Formula I, Y³, each Y¹, each Y² and each Y⁴ are all hydrogen, each Y⁵ is deuterium, R¹ and R² are the same, and the compound is selected from any one of the compounds set forth in Table 1c below:

TABLE 1c
Examples of Compounds of Formula I

| Compound | R¹ = R² | R³ | each Y⁶ | Y⁷ |
|---|---|---|---|---|
| 131 | CD₃ | Cl | D | D |
| 132 | CD₃ | Cl | H | D |
| 133 | CD₃ | CD₃ | D | D |
| 134 | CD₃ | CD₃ | H | D |
| 135 | CH₃ | Cl | D | D |
| 136 | CH₃ | Cl | H | D |
| 137 | CH₃ | CD₃ | D | D |
| 138 | CH₃ | CD₃ | H | D |

or a pharmaceutically acceptable salt thereof, wherein any atom not designated as deuterium is present at its natural isotopic abundance.

In one embodiment of the compound of Formula I, Y³, each Y¹, and each Y² are all hydrogen, each Y⁴ and each Y⁵ are deuterium, R¹ and R² are the same, and the compound is selected from any one of the compounds set forth in Table 1d below:

TABLE 1d
Examples of Compounds of Formula I

| Compound | R¹ = R² | R³ | each Y⁶ | Y⁷ |
|---|---|---|---|---|
| 141 | CD₃ | Cl | D | D |
| 142 | CD₃ | Cl | H | D |
| 143 | CD₃ | CD₃ | D | D |
| 144 | CD₃ | CD₃ | H | D |
| 145 | CH₃ | Cl | D | D |
| 146 | CH₃ | Cl | H | D |
| 147 | CH₃ | CD₃ | D | D |
| 148 | CH₃ | CD₃ | H | D |
| 151 | CD₃ | Cl | D | OH |
| 152 | CD₃ | Cl | H | OH |
| 153 | CD₃ | CD₃ | D | OH |
| 154 | CD₃ | CD₃ | H | OH |
| 155 | CH₃ | Cl | D | OH |
| 156 | CH₃ | Cl | H | OH |
| 157 | CH₃ | CD₃ | D | OH |
| 158 | CH₃ | CD₃ | H | OH |
| 161 | CD₃ | Cl | D | H |
| 162 | CD₃ | Cl | H | H |
| 163 | CD₃ | CD₃ | D | H |
| 164 | CD₃ | CD₃ | H | H |
| 165 | CH₃ | Cl | D | H |
| 166 | CH₃ | Cl | H | H |
| 167 | CH₃ | CD₃ | D | H |
| 168 | CH₃ | CD₃ | H | H |

or a pharmaceutically acceptable salt thereof, wherein any atom not designated as deuterium is present at its natural isotopic abundance.

In one embodiment, the compound of Formula A is a compound of Formula II

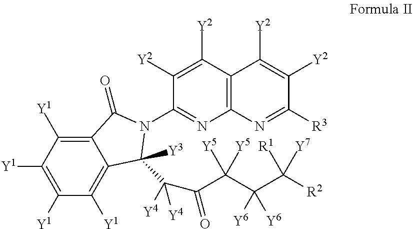

wherein Y¹, Y², Y³, Y⁴, Y⁵, Y⁶, Y⁷, R′, R² and R³ are each defined as in Formula A.

In one embodiment, the compound of Formula II is a compound of Formula IIa

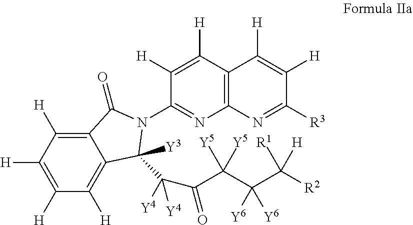

or a pharmaceutically acceptable salt thereof,
wherein

Y³, Y⁴, Y⁵, Y⁶, R¹, R² and R³ are each defined as in formula II, and wherein R¹ and R² are the same.

In one embodiment, the compound of Formula I is a compound of Formula IIb

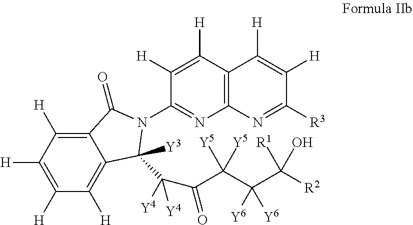

or a pharmaceutically acceptable salt thereof,
wherein

Y³, Y⁴, Y⁵, Y⁶, R¹, R² and R³ are each defined as in Formula II, and wherein R¹ and R² are the same.

In one embodiment, the compound of Formula II is a compound of the Formula IIc

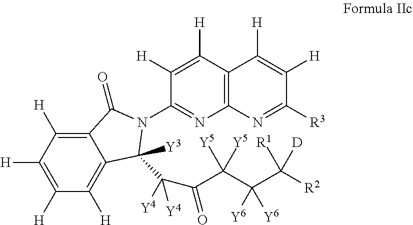

or a pharmaceutically acceptable salt thereof,
wherein:

Y³, Y⁴, Y⁵, Y⁶, R¹, R² and R³ are each defined as in Formula II, and wherein R¹ and R² are the same.

In one embodiment of Formula II or IIa or IIb or IIc, each Y⁶ is deuterium. In one aspect of this embodiment, R¹ and R² are each CD₃. In another aspect, R¹ and R² are each CH₃. In one aspect of this embodiment, R³ is Cl. In another aspect, R³ is CD₃. In one aspect of this embodiment, each Y⁵ is hydrogen. In another aspect, each Y⁵ is deuterium. In one aspect of this embodiment, each Y⁴ is hydrogen. In another aspect, each Y⁴ is deuterium.

In one embodiment of Formula II or IIa or IIb or IIc, R¹ and R² are each CD₃. In one aspect of this embodiment each Y⁶ is hydrogen. In one aspect of this embodiment, R³ is Cl. In another aspect, R³ is CD₃. In one aspect of this embodiment, each Y⁵ is hydrogen. In another aspect, each Y⁵ is deuterium. In one aspect of this embodiment, each Y⁴ is hydrogen. In another aspect, each Y⁴ is deuterium.

In another set of embodiments, any atom not designated as deuterium in any of the embodiments of Formula II, IIa, IIb or IIc set forth above is present at its natural isotopic abundance.

In one embodiment, the compound is a compound of Formula II wherein Y³, each Y¹, each Y², each Y⁴ and each Y⁵ are all hydrogen, R¹ and R² are the same, and the compound is selected from any one of the compounds set forth in Table 2a below.

TABLE 2a
Examples of Compounds of Formula II

| Compound | R¹ = R² | R³ | each Y⁶ | Y⁷ |
|---|---|---|---|---|
| 201 | CD₃ | Cl | D | OH |
| 202 | CD₃ | Cl | D | H |
| 203 | CD₃ | Cl | H | OH |
| 204 | CD₃ | Cl | H | H |
| 205 | CD₃ | CD₃ | D | OH |
| 206 | CD₃ | CD₃ | D | H |
| 207 | CD₃ | CD₃ | H | OH |
| 208 | CD₃ | CD₃ | H | H |
| 209 | CH₃ | Cl | D | OH |
| 210 | CH₃ | Cl | D | H |
| 211 | CH₃ | Cl | H | OH |
| 212 | CH₃ | CD₃ | D | OH |
| 213 | CH₃ | CD₃ | D | H |
| 214 | CH₃ | CD₃ | H | OH |
| 215 | CH₃ | CD₃ | H | H |

or a pharmaceutically acceptable salt thereof, wherein any atom not designated as deuterium is present at its natural isotopic abundance.

In one embodiment, the compound is a compound of Formula II wherein Y³, each Y¹, each Y², each Y⁴ and each Y⁵ are all hydrogen, R¹ and R² are the same, and the compound is selected from any one of the compounds set forth in Table 2b below.

TABLE 2b
Examples of Compounds of Formula II

| Compound | R¹ = R² | R³ | each Y⁶ | Y⁷ |
|---|---|---|---|---|
| 221 | CD₃ | Cl | D | D |
| 222 | CD₃ | Cl | H | D |
| 223 | CD₃ | CD₃ | D | D |
| 224 | CD₃ | CD₃ | H | D |
| 225 | CH₃ | Cl | D | D |
| 226 | CH₃ | Cl | H | D |
| 227 | CH₃ | CD₃ | D | D |
| 228 | CH₃ | CD₃ | H | D |

or a pharmaceutically acceptable salt thereof, wherein any atom not designated as deuterium is present at its natural isotopic abundance.

In one embodiment of the compound of Formula II, Y³, each Y¹, each Y² and each Y⁴ are all hydrogen, each Y⁵ is deuterium, R¹ and R² are the same, and the compound is selected from any one of the compounds set forth in Table 2c below:

TABLE 2c
Examples of Compounds of Formula II

| Compound | R¹ = R² | R³ | each Y⁶ | Y⁷ |
|---|---|---|---|---|
| 231 | CD₃ | Cl | D | D |
| 232 | CD₃ | Cl | H | D |
| 233 | CD₃ | CD₃ | D | D |
| 234 | CD₃ | CD₃ | H | D |
| 235 | CH₃ | Cl | D | D |
| 236 | CH₃ | Cl | H | D |
| 237 | CH₃ | CD₃ | D | D |
| 238 | CH₃ | CD₃ | H | D |

or a pharmaceutically acceptable salt thereof, wherein any atom not designated as deuterium is present at its natural isotopic abundance.

In one embodiment of the compound of Formula II, Y³, each Y¹, and each Y² are all hydrogen, each Y⁴ and each Y⁵ are deuterium, R¹ and R² are the same, and the compound is selected from any one of the compounds set forth in Table 2d below:

TABLE 2d
Examples of Compounds of Formula II

| Compound | R¹ = R² | R³ | each Y⁶ | Y⁷ |
|---|---|---|---|---|
| 241 | CD₃ | Cl | D | D |
| 242 | CD₃ | Cl | H | D |
| 243 | CD₃ | CD₃ | D | D |
| 244 | CD₃ | CD₃ | H | D |
| 245 | CH₃ | Cl | D | D |
| 246 | CH₃ | Cl | H | D |
| 247 | CH₃ | CD₃ | D | D |
| 248 | CH₃ | CD₃ | H | D |
| 251 | CD₃ | Cl | D | OH |
| 252 | CD₃ | Cl | H | OH |
| 253 | CD₃ | CD₃ | D | OH |
| 254 | CD₃ | CD₃ | H | OH |
| 255 | CH₃ | Cl | D | OH |
| 256 | CH₃ | Cl | H | OH |
| 257 | CH₃ | CD₃ | D | OH |
| 258 | CH₃ | CD₃ | H | OH |
| 261 | CD₃ | Cl | D | H |
| 262 | CD₃ | Cl | H | H |
| 263 | CD₃ | CD₃ | D | H |
| 264 | CD₃ | CD₃ | H | H |
| 265 | CH₃ | Cl | D | H |
| 266 | CH₃ | Cl | H | H |
| 267 | CH₃ | CD₃ | D | H |
| 268 | CH₃ | CD₃ | H | H |

or a pharmaceutically acceptable salt thereof, wherein any atom not designated as deuterium is present at its natural isotopic abundance.

In one embodiment of formula A, each Y⁶ is deuterium. In one aspect of this embodiment, R¹ and R² are each CD₃. In another aspect, R¹ and R² are each CH₃. In one aspect of this embodiment, R³ is Cl. In another aspect, R³ is CD₃. In one aspect of this embodiment, each Y⁵ is hydrogen. In another aspect, each Y⁵ is deuterium. In one aspect of this embodiment, each Y⁴ is hydrogen. In another aspect, each Y⁴ is deuterium.

In one embodiment of formula A, R¹ and R² are each CD₃. In one aspect of this embodiment each Y⁶ is hydrogen. In one aspect of this embodiment, R³ is Cl. In another aspect, R³ is CD₃. In one aspect of this embodiment, each Y⁵ is hydrogen. In another aspect, each Y⁵ is deuterium. In one aspect of this embodiment, each Y⁴ is hydrogen. In another aspect, each Y⁴ is deuterium.

In another set of embodiments, any atom not designated as deuterium in any of the embodiments of formula A set forth above is present at its natural isotopic abundance.

In one embodiment of the compound of Formula A, Y³, each Y¹, each Y², each Y⁴ and each Y⁵ are all hydrogen, R¹ and R² are the same, and the compound is a racemic mixture, wherein the compound is selected from any one of the compounds set forth in Table 3a below:

TABLE 3a
Examples of Compounds of Formula A

| Compound | R¹ = R² | R³ | each Y⁶ | Y⁷ |
|---|---|---|---|---|
| 301 | CD₃ | Cl | D | OH |
| 302 | CD₃ | Cl | D | H |
| 303 | CD₃ | Cl | H | OH |
| 304 | CD₃ | Cl | H | H |
| 305 | CD₃ | CD₃ | D | OH |
| 306 | CD₃ | CD₃ | D | H |
| 307 | CD₃ | CD₃ | H | OH |
| 308 | CD₃ | CD₃ | H | H |
| 309 | CH₃ | Cl | D | OH |
| 310 | CH₃ | Cl | D | H |
| 311 | CH₃ | Cl | H | OH |
| 312 | CH₃ | CD₃ | D | OH |
| 313 | CH₃ | CD₃ | D | H |
| 314 | CH₃ | CD₃ | H | OH |
| 315 | CH₃ | CD₃ | H | H |

or a pharmaceutically acceptable salt thereof, wherein any atom not designated as deuterium is present at its natural isotopic abundance.

In one embodiment of the compound of Formula A, Y³, each Y¹, each Y², each Y⁴ and each Y⁵ are all hydrogen, R¹ and R² are the same, the compound is a racemic mixture, and the compound is selected from any one of the compounds set forth in Table 3b below:

TABLE 3b
Examples of Compounds of Formula A

| Compound | R¹ = R² | R³ | each Y⁶ | Y⁷ |
|---|---|---|---|---|
| 321 | CD₃ | Cl | D | D |
| 322 | CD₃ | Cl | H | D |
| 323 | CD₃ | CD₃ | D | D |
| 324 | CD₃ | CD₃ | H | D |
| 325 | CH₃ | Cl | D | D |
| 326 | CH₃ | Cl | H | D |
| 327 | CH₃ | CD₃ | D | D |
| 328 | CH₃ | CD₃ | H | D |

or a pharmaceutically acceptable salt thereof, wherein any atom not designated as deuterium is present at its natural isotopic abundance.

In one embodiment of the compound of Formula A, Y³, each Y¹, each Y² and each Y⁴ are all hydrogen, each Y⁵ is deuterium, R³ and R² are the same, the compound is a racemic mixture, and the compound is selected from any one of the compounds set forth in Table 3c below:

TABLE 3c
Examples of Compounds of Formula A

| Compound | R¹ = R² | R³ | each Y⁶ | Y⁷ |
|---|---|---|---|---|
| 331 | CD₃ | Cl | D | D |
| 332 | CD₃ | Cl | H | D |
| 333 | CD₃ | CD₃ | D | D |
| 334 | CD₃ | CD₃ | H | D |
| 335 | CH₃ | Cl | D | D |
| 336 | CH₃ | Cl | H | D |
| 337 | CH₃ | CD₃ | D | D |
| 338 | CH₃ | CD₃ | H | D |

or a pharmaceutically acceptable salt thereof, wherein any atom not designated as deuterium is present at its natural isotopic abundance.

In one embodiment of the compound of Formula A, Y³, each Y¹, and each Y² are all hydrogen, each Y⁴ and each Y⁵ are deuterium, R¹ and R² are the same, the compound is a racemic mixture, and the compound is selected from any one of the compounds set forth in Table 3d below:

TABLE 3d
Examples of Compounds of Formula A

| Compound | R¹ = R² | R³ | each Y⁶ | Y⁷ |
|---|---|---|---|---|
| 341 | CD₃ | Cl | D | D |
| 342 | CD₃ | Cl | H | D |
| 343 | CD₃ | CD₃ | D | D |
| 344 | CD₃ | CD₃ | H | D |
| 345 | CH₃ | Cl | D | D |
| 346 | CH₃ | Cl | H | D |
| 347 | CH₃ | CD₃ | D | D |
| 348 | CH₃ | CD₃ | H | D |
| 351 | CD₃ | Cl | D | OH |
| 352 | CD₃ | Cl | H | OH |
| 353 | CD₃ | CD₃ | D | OH |
| 354 | CD₃ | CD₃ | H | OH |
| 355 | CH₃ | Cl | D | OH |
| 356 | CH₃ | Cl | H | OH |
| 357 | CH₃ | CD₃ | D | OH |
| 358 | CH₃ | CD₃ | H | OH |
| 361 | CD₃ | Cl | D | H |
| 362 | CD₃ | Cl | H | H |
| 363 | CD₃ | CD₃ | D | H |
| 364 | CD₃ | CD₃ | H | H |
| 365 | CH₃ | Cl | D | H |
| 366 | CH₃ | Cl | H | H |
| 367 | CH₃ | CD₃ | D | H |
| 368 | CH₃ | CD₃ | H | H |

The synthesis of compounds of Formula A, Formula I (including any of the formulae herein) or Formula II (including any of the formulae herein) can be readily achieved by synthetic chemists of ordinary skill following the Exemplary Synthesis and Examples disclosed herein. Other relevant procedures and intermediates are disclosed, for instance, in U.S. Pat. No. 4,960,779, in U.S. Pat. No. 5,494,915, and in Stuk, T. L.; et al. Org Proc Res Dev 2003, 7, 851-855.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Certain intermediates can be used with or without purification (e.g., filtration, distillation, sublimation, crystallization, trituration, solid phase extraction, and chromatography).

### Exemplary Synthesis

In the following Exemplary Syntheses, deuterated reagents and solvents may be substituted where appropriate to further optimize the isotopic purity of the desired products.

A convenient method for synthesizing compounds of Formula A, Formula I (including any of the formulae herein) or Formula II (including any of the formulae herein) wherein R³ is Cl is depicted in Scheme 1.

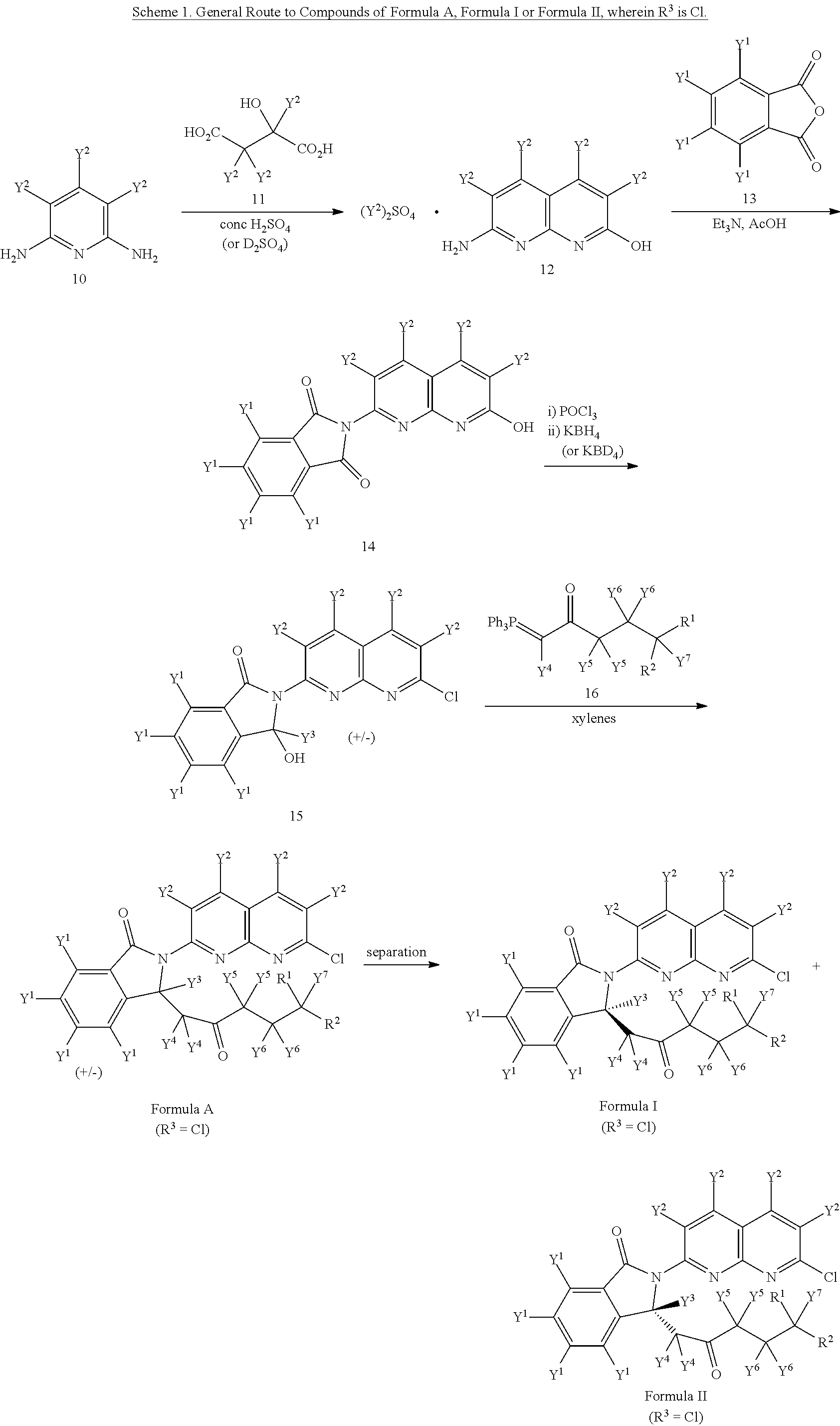

Scheme 1 depicts a general route to preparing compounds of Formula A, I or II wherein R³ is Cl. In a manner analogous to that described by Stuk, T. L.; et al. Org Proc Res Dev 2003, 7, 851-855, appropriately deuterated amine 10 is reacted with appropriately deuterated malic acid 11 and either H₂SO₄ or D₂SO₄ to afford sulfuric acid salt 12. Salt 12 is treated with appropriately deuterated phthalic anhydride 13 in the presence of triethylamine and glacial acetic acid to provide phthalimide 14. Compound 14 is treated with POCl₃ and then with KBH₄ (for compounds wherein Y³ is H) or KBD₄ (for compounds wherein Y³ is D; see Atkinson, J. G.; et al. Canadian Journal of Chemistry (1967), 45(21), 2583-8) to afford 15. Treatment of 15 with ylide 16 (see Scheme 2) affords compounds of Formula A wherein R³ is Cl. Several methods for converting racemic compounds of Formula A to compounds of Formula I and compounds of Formula II are available in the literature, including chiral chromatography as described by Stuk, T. L.; et al. Org Proc Res Dev 2003, 7, 851-855.

Useful commercially available examples of malic acid 11 include DL-malic acid (11a) and DL-malic acid-2,3,3-d3 (11b).

Useful commercially available examples of phthalic anhydride 13 include phthalic anhydride (13a) and phthalic-d4 anhydride (13b).

A convenient method for synthesizing ylide 16 is depicted in Scheme 2.

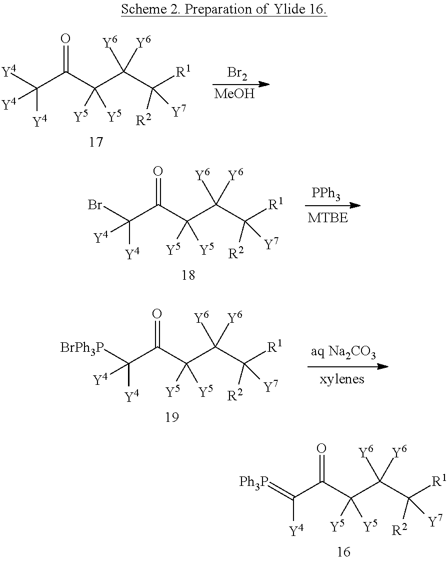

Scheme 2 depicts the preparation of appropriately deuterated ylide 16 in a manner analogous to that described by Stuk, T. L.; et al. Org Proc Res Dev 2003, 7, 851-855. Appropriately deuterated ketone 17 (see Scheme 3) is treated with bromine in methanol to afford bromoketone 18. Treatment with PPh₃ affords phosphonium salt 19. Treatment of 19 with aqueous Na₂CO₃ affords ylide 16, which is a useful intermediate for Scheme 1.

A convenient method for synthesizing appropriately deuterated ketone 17 is depicted in Scheme 3.

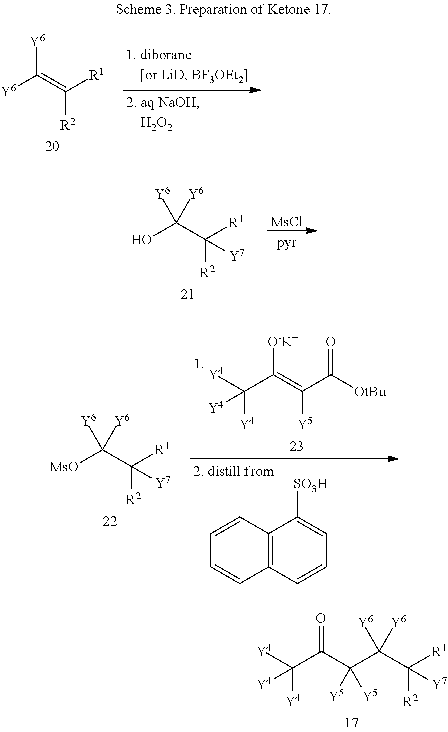

Scheme 3 depicts the preparation of appropriately deuterated ketone 17 in a manner analogous to that described by Wolff, S.; et al. J Am Chem Soc (1972), 94(22), 7797-7806. Appropriately deuterated isobutylene 20 is treated with either diborane (for compounds wherein Y⁷ is H) or deuterated diborane generated in situ from lithium deuteride and BF₃OEt₂ (for compounds wherein Y⁷ is D) to provide appropriately deuterated alcohol 21. Treatment of 21 with methanesulfonyl chloride and pyridine affords mesylate 22. Treatment with the potassium salt of appropriately deuterated tert-butyl acetoacetate (23) followed by distillation from naphthalene-1-sulfonic acid provides appropriately deuterated ketone 17, which is a useful intermediate for Scheme 2.

Useful commercially available examples of isobutylene 20 include the following:

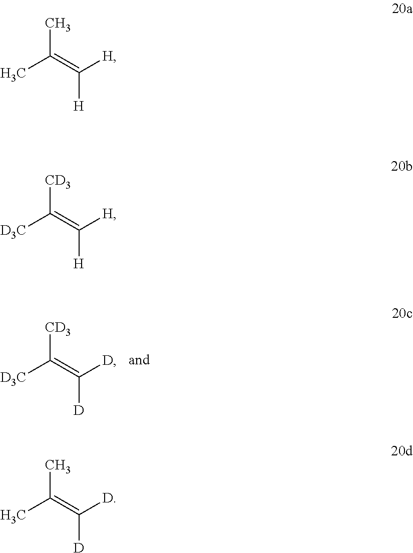

Potassium salt 23 is prepared from appropriately deuterated tert-butyl acetoacetate according to the method described in Wolff, S.; et al. J Am Chem Soc (1972), 94(22), 7797-7806. Useful examples of appropriately deuterated tert-butyl acetoacetate include

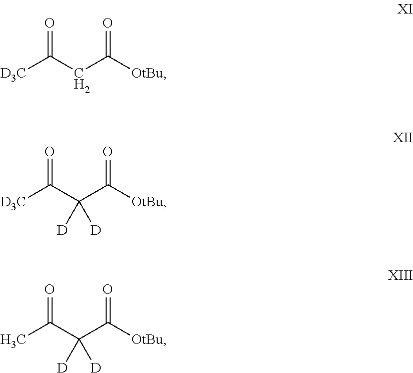 [SMILES: [2H]C([2H])([2H])C(=O)CC(=O)OC(C)(C)C ; [2H]C([2H])([2H])C(=O)C([2H])([2H])C(=O)OC(C)(C)C ; [2H]C([2H])(C(C)=O)C(=O)OC(C)(C)C]

or commercially available

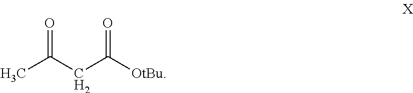

Compounds XI, XII, and XIII may be prepared from beta-ketoesters 28b, 28c, and 28d (see Scheme 5) and either tBuOH or tBuOD in a manner analogous to that described in either Bandgar, B. P.; et al. Journal of the Chinese Chemical Society (Taipei, Taiwan) (2005), 52(6), 1101-1104; or in Tale, R. H.; et al. Synlett (2006), (3), 415-418.

A convenient method for synthesizing compounds of Formula A, Formula I (including any of the formulae herein) or Formula II (including any of the formulae herein) wherein R³ is Cl and Y⁷ is OH is depicted in Scheme 4.

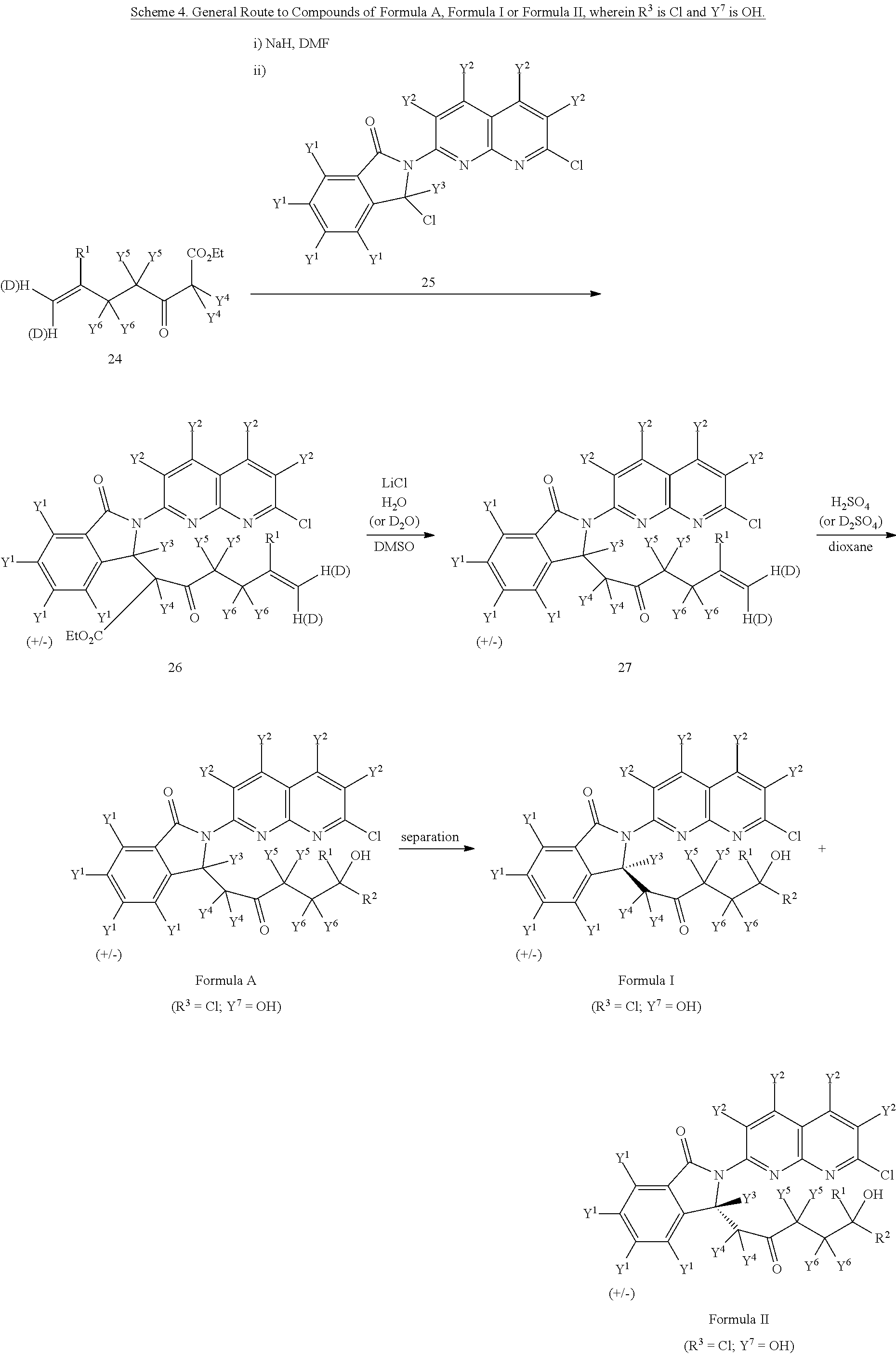

Scheme 4 depicts a general route to preparing compounds of Formula A, I or II wherein R³ is Cl and Y⁷ is OH. In a manner analogous to that described in U.S. Pat. No. 5,494,915, appropriately deuterated ketone 24 (see Scheme 5) is treated with NaH, followed by appropriately deuterated 25 (see Scheme 6) to afford ketone 26. Treatment of 26 with LiCl and either H₂O or D₂O in DMSO provides appropriately deuterated 27. Treatment of 27 with H₂SO₄ or D₂SO₄ in dioxane affords compounds of Formula A wherein R³ is Cl and Y⁷ is OH. Chiral chromatography as described in U.S. Pat. No. 5,494,915 converts racemic compounds of Formula A, wherein R³ is Cl and Y⁷ is OH, to compounds of Formula I and compounds of Formula II (wherein R³ is Cl and Y⁷ is OH).

A convenient method for synthesizing ketone 24 is depicted in Scheme 5.

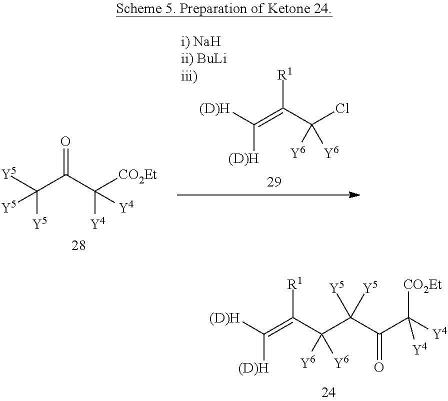

Scheme 5 depicts the preparation of appropriately deuterated ketone 24 in a manner analogous to that described by Rao, V. B.; et al. JACS 1985, 107, 5732. Appropriately deuterated beta-ketoester 28 is treated sequentially with NaH and BuLi, followed by alkylation with appropriately deuterated halide 29 to afford ketone 24, which is a useful intermediate for Scheme 4.

Useful examples of beta-ketoester 28 include commercially available CH₃C(O)CH₂CO₂Et (28a); known CD₃C(O)CD₂CO₂Et (28b) [see Guengerich, F. P.; et al. Journal of Biological Chemistry (1988), 263(17), 8176-83; and Duguay, G.; et al. Bulletin de la Societe Chimique de France (1974), (12, Pt. 2), 2853-6]; and known CH₃C(O)CD₂CO₂Et (28c) [see Thulasiram, H. V.; et al. Journal of Organic Chemistry (2006), 71(4), 1739-1741]. Additionally, methyl ester CD₃C(O)CH₂CO₂Me (28d) is known [see Tortajada, J.; et al. J. Am. Chem. Soc. 1992, 114, 10874-10880] and may be used similarly in Scheme 5 as beta-ketoester 28.

Useful examples of halide 29 include commercially available

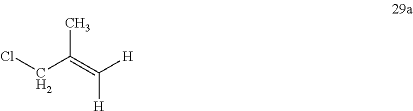

and known

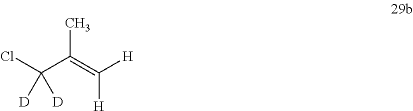

(see Anisimov, A. V.; et al. Zhurnal Organicheskoi Khimii (1981), 17(6), 1316-19). Additionally, useful halide

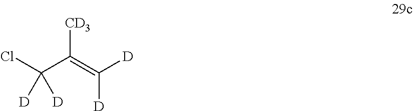 [SMILES: [2H]C([2H])=C(C([2H])([2H])[2H])C([2H])([2H])Cl]

may be prepared from commercially available 2-methylpropene-d8 and Cl₂ in a manner analogous to that described in Schulze, K.; et al. Journal fuer Praktische Chemie (Leipzig) (1984), 326(3), 433-42; or in Chinese patent application CN 101182279.

A convenient method for synthesizing appropriately deuterated intermediate 25 is depicted in Scheme 6.

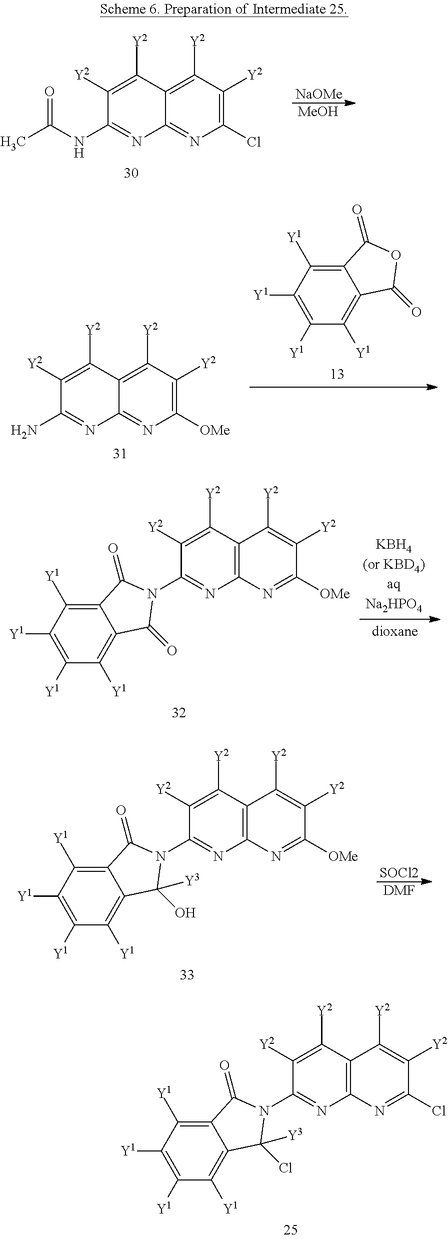

Scheme 6 depicts the preparation of appropriately deuterated 25 in a manner analogous to that described in Belgian Patent No. 815019. Appropriately deuterated amide 30 is treated with NaOMe in MeOH to afford amine 31. Treatment of 31 with appropriately deuterated phthalic anhydride provides 32. Reduction with either KBH₄ or KBD₄ (see Atkinson, J. G.; et al. Canadian Journal of Chemistry (1967), 45(21), 2583-8) provides appropriately deuterated 33. Treatment of 33 with SOCl₂ affords intermediate 25, which is a useful intermediate for Scheme 4.

Appropriately deuterated amide 30 is prepared from appropriately deuterated malic acid 11 and from appropriately deuterated 2,6-diaminopyridine 10 in a manner analogous to that described in Carboni, S.; et al. Gazz. Chim. It. 1965, 95, 1492-1501, and in Anderson, C. A.; et al. Journal of Organic Chemistry (2010), 75(14), 4848-4851.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula A, Formula I (including any of the formulae herein) or Formula II (including any of the formulae herein) and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free compositions comprising an effective amount of a compound of Formula A, Formula I (including any of the formulae herein) or Formula II (including any of the formulae herein), or a pharmaceutically acceptable salt of said compound; and an acceptable carrier. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

In another embodiment, a composition of this invention further comprises a second therapeutic agent. The second therapeutic agent may be selected from any compound or therapeutic agent known to have or that demonstrates advantageous properties when administered with a compound having the same mechanism of action as pagoclone.

Preferably, the second therapeutic agent is an agent useful in the treatment of a disease or condition such as a disorder of the central nervous system, including anxiety, including general anxiety disorder and social anxiety disorder; agoraphobia; attention deficit hyperactivity disorder (ADHD); autism; bipolar disorder, including bipolar I disorder and bipolar II disorder; dementia, including dementia due to Parkinson's disease and dementia of the Alzheimer's type; insomnia; major depressive disorder; narcolepsy; obsessive-compulsive disorder (OCD); panic disorder, including panic disorder with agoraphobia and panic disorder without agoraphobia; post-traumatic stress disorder (PTSD); schizophrenia; sleep disorder; social phobia; stuttering; Tourette's disorder; epilepsy, seizures, and/or convulsions; neuropathic, inflammatory and migraine associated pain; and premature ejaculation. In one embodiment, the disorders include anxiety, such as general anxiety disorder and social anxiety; panic disorder; epilepsy, seizures, and/or convulsions; neuropathic, inflammatory and migraine associated pain; and premature ejaculation. The agent may be, for example, an anxiolytic, hypnotic, anticonvulsant, antiepileptic or muscle relaxant.

In one embodiment the second therapeutic agent is an agent useful in the treatment of a disease or condition such as anxious depression

In one embodiment the second therapeutic agent is an agent useful in the treatment of a disease or condition such as spasticity and conditions related to spasticity, such as overactive bladder or interstitial cystitis.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 0.01 to about 5000 mg per treatment. In more specific embodiments the range is from about 0.1 to 2500 mg, or from 0.2 to 1000 mg, or most specifically from about 1 to 500 mg. Treatment typically is administered one to three times daily.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of modulating the GABA-A receptor in a cell, comprising contacting a cell with one or more compounds of Formula I (including any of the formulae herein) or Formula II (including any of the formulae herein) herein or a salt thereof.

According to another embodiment, the invention provides a method of treating in a subject, such as a patient, in need of such treatment, a disease that is beneficially treated by pagoclone comprising the step of administering to said subject an effective amount of a compound of Formula A, Formula I (including any of the formulae herein) or Formula II (including any of the formulae herein) or a pharmaceutically acceptable salt thereof, or a composition of this invention. Such diseases include disorders of the central nervous system, including anxiety, including general anxiety disorder and social anxiety disorder; agoraphobia; attention deficit hyperactivity disorder (ADHD); autism; bipolar disorder, including bipolar I disorder and bipolar II disorder; dementia, including dementia due to Parkinson's disease and dementia of the Alzheimer's type; insomnia; major depressive disorder; narcolepsy; obsessive-compulsive disorder (OCD); panic disorder, including panic disorder with agoraphobia and panic disorder without agoraphobia; post-traumatic stress disorder (PTSD); schizophrenia; sleep disorder; social phobia; stuttering; Tourette's disorder; epilepsy, seizures, and/or convulsions; neuropathic, inflammatory and migraine associated pain; and premature ejaculation. In one embodiment, the disorders include anxiety, such as general anxiety disorder and social anxiety; panic disorder; epilepsy, seizures, and/or convulsions; neuropathic, inflammatory and migraine associated pain; and premature ejaculation. The compound of Formula A, Formula I or Formula II may be used, for example, as an anxiolytic, hypnotic, anticonvulsant, antiepileptic or muscle relaxant.

In one embodiment, such diseases include anxious depression.

In one embodiment, such diseases include spasticity and conditions related to spasticity, such as overactive bladder or interstitial cystitis.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to said patient one or more second therapeutic agents. The choice of second therapeutic agent may be made from any second therapeutic agent known to be useful for co-administration with pagoclone. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

### EXAMPLES

### Example 1

### Synthesis of (+/−)-2-(7-Chloro-1,8-naphthyridin-2-yl)-3-(5-(d₃-methyl)-2-oxo-3,3,4,4,5,6,6,6-d₈-hexyl)isoindolin-1-one (Compound 131 and its (−)enantiomer)

A racemic mixture of Compound 131 and its (−)enantiomer was prepared as outlined in Scheme 7 below.

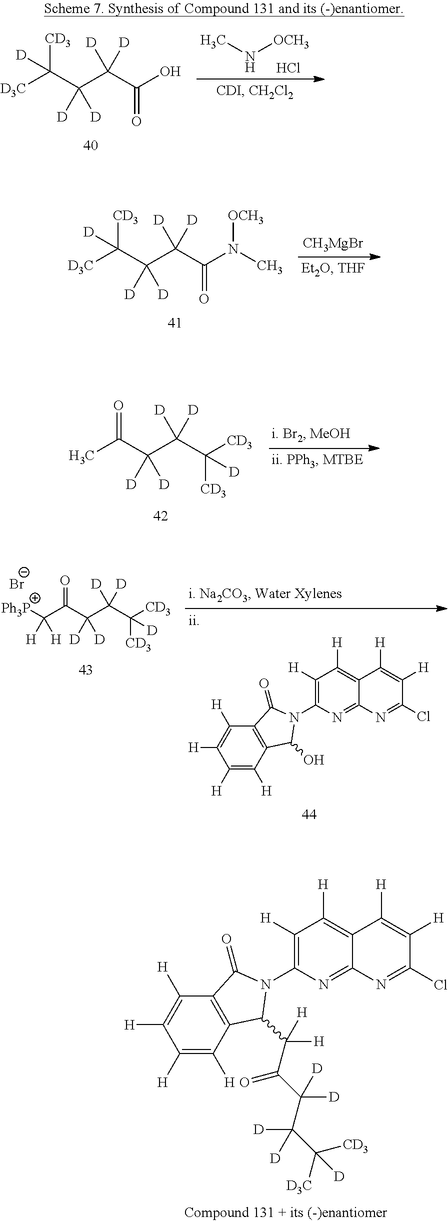

### Step 1. N-Methoxy-N-methyl-4-(d₃-methyl)-2,2,3,3,4,5,5,5-d₈-pentanamide (41)

To a solution of 4-methylpenatnoic acid-d11, 40 (1.00 g, 7.86 mmol, CDN Isotopes, 98 atom % D) in dichloromethane (16 mL) was added 1,1′ carbonyldiimidazole (1.27 g, 7.86 mmol). After stirring at room temperature for 15 minutes, N,O-dimethylhydroxylamine hydrochloride (767 mg, 7.86 mmol) was added and stirring at room temperature was continued for 1 hour. The reaction was then quenched with 1N HCl (9 mL), diluted with water and extracted with diethyl ether (3×25 mL). The organic layers were combined, washed with saturated NaHCO₃, dried (MgSO₄), filtered and concentrated under reduced pressure to afford 41 as a clear oil (1.23 g, 92%). ¹H NMR (CDCl₃, 400 MHz) δ 3.68 (s, 3H), 3.17 (s, 3H). MS (ESI) 171.4 [(M+H)⁺].

### Step 2. 5-(d₃-Methyl)-3,3,4,4,5,6,6,6-d₈-hexan-2-one (42)

To a solution of pentanamide 41 (3.76 g, 22.1 mmol) in THF (90 mL) at 0° C. was added a 3M solution of methylmagnesium bromide in diethylether (11.0 mL, 33.1 mmol). The reaction was stirred at room temperature for 15 hours then was cooled to 0° C. and quenched with 1N HCl. The resulting solution was extracted with diethyl ether (3×50 mL), dried (MgSO₄), filtered and concentrated under reduced pressure to afford 42 as a light yellow oil (2.48 g, 90%) which was used without further purification. ¹H NMR (CDCl₃, 400 MHz) δ 2.14 (s, 3H).

### Step 3. (5-(d₃-Methyl)-2-oxo-3,3,4,4,5,6,6,6-d₈-hexyl)triphenylphosphonium bromide (43)

To a solution of pentanone 42 (2.48 g, 19.8 mmol) in methanol (20 mL) at 0° C. was added bromine (883 μL, 17.2 mmol). The reaction was stirred at 10° C. for 2 hours then was quenched with water (3.5 mL) and stirring was continued for an additional 30 minutes. The reaction was then diluted with MTBE (50 mL), washed with NaHCO₃ followed by brine, dried (Na₂SO₄), filtered and concentrated. The resulting residue was repeatedly dissolved in MTBE and concentrated under vacuum (3×) to remove residual methanol. The residue was then taken up in MTBE (5 mL), cooled to 10° C. and a solution of triphenylphosphine (4.51 g, 17.2 mmol) in MTBE (5 mL) was added (in order to maintain solubility, the triphenylphosphine/MTBE solution required warming). After stirring at room temperature for 15 hours, the precipitate was filtered, rinsed with MTBE (3×5 mL) and dried under vacuum to provide 43 as a white solid (3.31 g, 36%). ¹H NMR (CDCl₃, 400 MHz) δ 7.92-7.63 (m, 15H), 5.60 (d, J=12.8 Hz, 2H). MS (ESI) 386.3 [(M−Br)⁺].

### Step 4. (+/−)-2-(7-Chloro-1,8-naphthyridin-2-yl)-3-(5-(d₃-methyl)-2-oxo-3,3,4,4,5,6,6,6-d₈-hexyl)isoindolin-1-one (Compound 131 and its (−)enantiomer)

To a suspension of triphenylphosphonium bromide 43 (1.21 g, 2.59 mmol) in a mixture of water (6 mL) and xylenes (6 mL) was added Na₂CO₃ (576 mg, 5.43 mmol). After stirring at room temperature for 30 minutes the aqueous layer was removed and the organic layer was washed with 5% Na₂CO₃ (3×5 mL). The organic layer was then added to rac-2-(7-chloro-1,8-naphthyridin-2-yl)-3-hydroxyisoindolin-1-one (44, preparation described in Org. Process Res. Dev. 2003, 7, 851-855) (504 mg, 1.62 mmol) and the reaction was stirred at reflux for 24 hours (residual water was removed during the course of the reaction through the use of a Dean-Stark trap). At this time the reaction was cooled to 80° C. and the majority of the xylenes was removed via distillation. 2-Propanol (10 mL) was then added to the resulting slurry and the reaction was stirred at reflux for 15 minutes to dissolve all solids. The reaction was then cooled to room temperature and the resulting precipitate was filtered and subsequently rinsed with additional 2-propanol (2×5 mL) followed by methanol (2×5 mL). The resulting material was then dried under vacuum to afford Compound 131 and its (−)enantiomer as a racemic mixture (468 mg, 69%) as a light pink solid. MS (ESI) 419.2 [(M+H)⁺].

### Example 2

### Synthesis of (+)-2-(7-Chloro-1,8-naphthyridin-2-yl)-3-(5-(d₃-methyl)-2-oxo-3,3,4,4,5,6,6,6-d₈-hexyl)isoindolin-1-one (Compound 131).

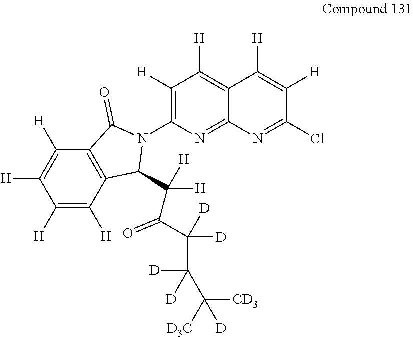 [SMILES: [2H]C([2H])([2H])C([2H])(C([2H])([2H])[2H])C([2H])([2H])C([2H])([2H])C(=O)C[C@H]1c2ccccc2C(=O)N1c1ccc2ccc(Cl)nc2n1]

### (+)-2-(7-Chloro-1,8-naphthyridin-2-yl)-3-(5-(d₃-methyl)-2-oxo-3,3,4,4,5,6,6,6-d₈-hexyl)isoindolin-1-one (Compound 131)

Optically pure Compound 131 is obtained either through chiral separation or chemical resolution of the racemic mixture of Compound 131 and its (−)enantiomer, both of which have been previously described for pagoclone (Org. Process Res. Dev. 2003, 7, 851-855).

### Example 3

### Synthesis of (+/−)-2-(7-Chloro-1,8-naphthyridin-2-yl)-3-(5-(d₃-methyl)-2-oxo-4,4,5,6,6,6-d₆-hexyl)isoindolin-1-one (Compound 121 and its (−)enantiomer)

A racemic mixture of Compound 121 and its (−)enantiomer was prepared as outlined in Scheme 8 below.

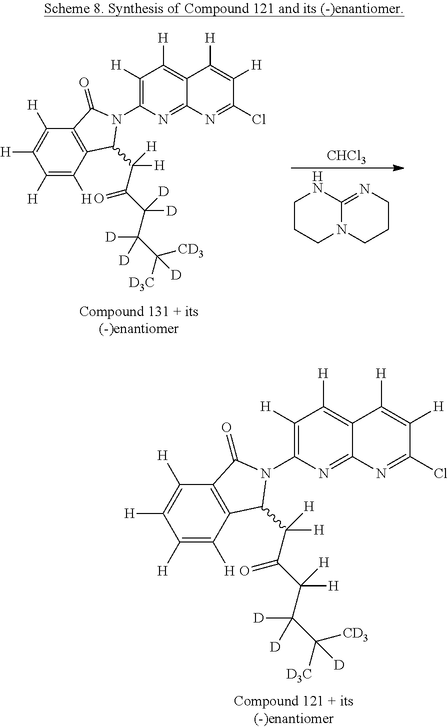 [SMILES: [2H]C([2H])([2H])C([2H])(C([2H])([2H])[2H])C([2H])([2H])C([2H])([2H])C(=O)CC1c2ccccc2C(=O)N1c1ccc2ccc(Cl)nc2n1 ; C1CN=C2NCCCN2C1 ; "CHCl3" ; [2H]C([2H])([2H])C([2H])(C([2H])([2H])[2H])C([2H])([2H])CC(=O)CC1c2ccccc2C(=O)N1c1ccc2ccc(Cl)nc2n1]

### (+/−)-2-(7-Chloro-1,8-naphthyridin-2-yl)-3-(5-(d₃-methyl)-2-oxo-4,4,5,6,6,6-d₆-hexyl)isoindolin-1-one (Compound 121 and its (−)enantiomer)

To a solution of Compound 131 and its (−) enantiomer (50 mg, 0.12 mmol) in chloroform (2.0 mL) was added 1,5,7-triazabicyclo[4.4.0]dec-5-ene (5.0 mg, 0.035 mmol). After stirring at room temperature for 24 hours, the reaction was then quenched with 1N HCl and extracted with chloroform (3×5 mL). The organic layers were combined, washed with, brine, dried (Na₂SO₄), filtered and concentrated under reduced pressure to afford a racemic mixture of Compound 121 and its (−)enantiomer. MS (ESI) 417.3 [(M+H)⁺].

### Example 4

### Synthesis of (+)-2-(7-Chloro-1,8-naphthyridin-2-yl)-3-(5-(d₃-methyl)-2-oxo-4,4,5,6,6,6-d₆-hexyl)isoindolin-1-one (Compound 121)

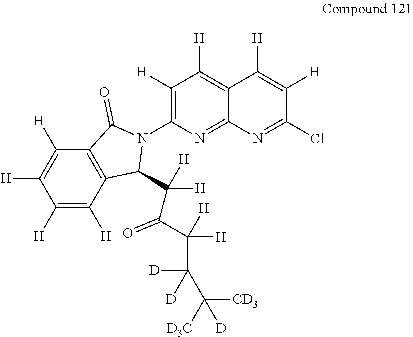 [SMILES: [2H]C([2H])([2H])C([2H])(C([2H])([2H])[2H])C([2H])([2H])CC(=O)C[C@H]1c2ccccc2C(=O)N1c1ccc2ccc(Cl)nc2n1]

### (+)-2-(7-Chloro-1,8-naphthyridin-2-yl)-3-(5-(d₃-methyl)-2-oxo-4,4,5,6,6,6-d₆=hexyl)isoindolin-1-one (Compound 121)

Optically pure Compound 121 is obtained either through chiral separation or chemical resolution of the racemic mixture of Compound 121 and its (−)enantiomer, both of which have been previously described for pagoclone (Org. Process Res. Dev. 2003, 7, 851-855).

### Example 5

### Synthesis of (+/−)-2-(7-Chloro-1,8-naphthyridin-2-yl)-3-(5-(d₃-methyl)-2-oxo-1,1,3,3,4,4,5,6,6,6-d₁₀-hexyl)isoindolin-1-one (Compound 141 and its (−)enantiomer)

A racemic mixture of Compound 141 and its (−)enantiomer was prepared as outlined in Scheme 9 below.

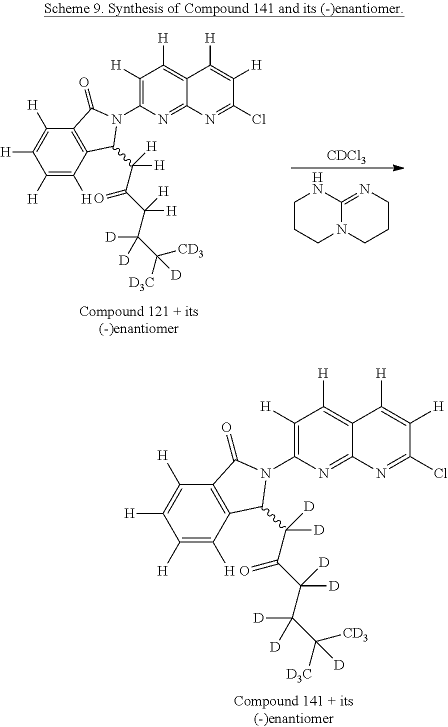 [SMILES: [2H]C([2H])([2H])C([2H])(C([2H])([2H])[2H])C([2H])([2H])CC(=O)CC1c2ccccc2C(=O)N1c1ccc2ccc(Cl)nc2n1 ; C1CN=C2NCCCN2C1 ; "CDCl3" ; [2H]C([2H])(C(=O)C([2H])([2H])C([2H])([2H])C([2H])(C([2H])([2H])[2H])C([2H])([2H])[2H])C1c2ccccc2C(=O)N1c1ccc2ccc(Cl)nc2n1]

### (+/−)-2-(7-Chloro-1,8-naphthyridin-2-yl)-3-(5-(d₃-methyl)-2-oxo-4,4,5,6,6,6-d₆-hexyl)isoindolin-1-one (Compound 141 and its (−)enantiomer)

To a solution of Compound 121 and its (−) enantiomer (41 mg, 0.098 mmol) in CDCl₃ (3.0 mL) was added 1,5,7-triazabicyclo[4.4.0]dec-5-ene (4.0 mg, 0.029 mmol). After stirring at room temperature for 24 hours, the reaction was then quenched with 1N HCl and extracted with chloroform (3×5 mL). The organic layers were combined, washed with, brine, dried (Na₂SO₄), filtered and concentrated under reduced pressure to afford a racemic mixture of Compound 141 and its (−)enantiomer. MS (ESI) 421.3 (M+H)⁺].

### Example 6

### Synthesis of (+/−)-2-(7-Chloro-1,8-naphthyridin-2-yl)-3-(5-(d₃-methyl)-2-oxo-1,1,3,3,4,4,5,6,6,6-d₁₀-hexyl)isoindolin-1-one (Compound 141)

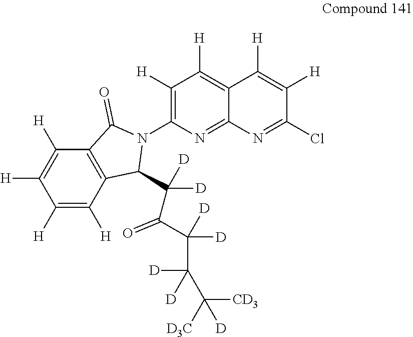 [SMILES: [2H]C([2H])(C(=O)C([2H])([2H])C([2H])([2H])C([2H])(C([2H])([2H])[2H])C([2H])([2H])[2H])[C@H]1c2ccccc2C(=O)N1c1ccc2ccc(Cl)nc2n1]

### (+/−)-2-(7-Chloro-1,8-naphthyridin-2-yl)-3-(5-(d₃-methyl)-2-oxo-1,1,3,3,4,4,5,6,6,6-d₁₀-hexyl)isoindolin-1-one (Compound 141)

Optically pure Compound 141 is obtained either through chiral separation or chemical resolution of the racemic mixtue of Compound 141 and its (−)enantiomer, both of which have been previously described for pagoclone (Org. Process Res. Dev. 2003, 7, 851-855).

### Example 7

### Evaluation of Metabolic Stability

Microsomal Assay: Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability: 7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Incubations are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-deuterated counterpart of the compound of Formula I and the positive control, 7-ethoxycoumarin (1 μM). Testing is done in triplicate.

Data analysis: The in vitro tᵤ₂s for test compounds are calculated from the slopes of the linear regression of % parent remaining (1n) vs incubation time relationship.

in vitro t_{1/2}=0.693/k

k=−[slope of linear regression of % parent remaining(ln) vs incubation time]

Data analysis is performed using Microsoft Excel Software.

Hepatocyte Assay: Pagoclone or a compound of Formula A, Formula I (including any of the formulae herein) or Formula II (including any of the formulae herein) is incubated with human hepatocyes at concentration of 10-25 uM up to 4 hrs. Reactions are stopped by the addition of acetonitrile and samples are centrifuged to remove precipitated proteins and cell debris. Supernatants are analyzed for metabolites/metabolite profiles by HPLC-UV or LC-MS/MS analyses.

In vivo Assay using rats: Male Sprague-Dawley rats are dosed IV or PO at 1 or 3 mg/kg in an appropriate dosing vehicle with a compound of Formula A, Formula I (including any of the formulae herein) or Formula II (including any of the formulae herein). Blood sample is drawn at pre-dose and approximately 8 time-points post-dose. Plasma is harvested from the blood samples, and is analyzed for amounts of dosed parent, and optionally for metabolites, by LC-MS/MS. PK analyses are performed by Non-Compartmental analyses using WinNonlin.

### Example 8

### Determination of Metabolic stability in Human Liver S9 Fractions

A. Materials and Methods:

Materials: Human liver S9 (20 mg/mL) were obtained from Xenotech, LLC (Lenexa, Kans.). (3-nicotinamide adenine dinucleotide phosphate, reduced form (NADPH), magnesium chloride (MgCl₂), and dimethyl sulfoxide (DMSO) were purchased from Sigma-Aldrich.

Determination of Metabolic Stability: 10 mM stock solutions of test compounds were prepared in DMSO. The 10 mM stock solutions were diluted to 0.1 mM or 1.0 mM in acetonitrile (ACN). The 20 mg/mL human liver microsomes were diluted to 2.5 mg/mL in 0.1 M potassium phosphate buffer, pH 7.4, containing 3 mM MgCl₂ and incubated with alamethicin at 10 μg/mL for 15 minutes on ice. The diluted S9 were added to wells of a 96-well deep-well polypropylene plate in triplicate. 15 μL of the 0.1 mM or 1.0 mM test compound was added to the S9 and the mixture was pre-warmed for 10 minutes. Reactions were initiated by addition of pre-warmed NADPH solution. The final reaction volume was 0.5 mL and contained 2.0 mg/mL human liver S9, 0.75 μM or 7.5 μM test compound, and 2 mM NADPH in 0.1 M potassium phosphate buffer, pH 7.4, and 3 mM MgCl₂. The reaction mixtures were incubated at 37° C., and 50 μL aliquots were removed at 0 and 60 minutes and added to shallow-well 96-well plates which contained 50 μL of ice-cold ACN with internal standard to stop the reactions. The plates were stored at 4° C. for 20 minutes after which 100 μL of water was added to the wells of the plate before centrifugation to pellet precipitated proteins. Supernatants were transferred to another 96-well plate and analyzed for amounts of parent remaining by LC-MS/MS using an Applied Bio-systems API 4000 mass spectrometer.

Data analysis: The in vitro tᵤ₂s for test compounds were calculated from the slopes of the linear regression of % parent remaining (1n) vs incubation time relationship.

in vitro t_{1/2}=0.693/k

k=−[slope of linear regression of % parent remaining(ln) vs incubation time]

Data analysis was performed using Microsoft Excel Software.

B. Results

Pagoclone, (S)-pagoclone, and compound rac-141 were tested in human liver S9 fractions according to the above method. The results of the assay for each of the three compounds are shown in Tables 4a and 4b.

TABLE 4a
% parent compound remaining after 1 hr in human liver S9 fractions

| | % Parent remaining at 1 hr | |
|---|---|---|
| 1 μM tested compound | % Δ | |
| Pagoclone (racemic) | 37 ± 3.8 | |
| Pagoclone (S) | 41 ± 1.0 | |
| Rac-141 | 60 ± 8.4 | |
| | 62% | (relative to racemic) |
| | 46% | (relative to S) |

TABLE 4b
% parent compound remaining after 1 hr in human liver S9 fractions

| | % Parent remaining at 1 hr | |
|---|---|---|
| 10 μM tested compound | % Δ | |
| 1 Pagoclone (racemic) | 11 ± 1.2 | |
| Pagoclone (S) | 23 ± 3.9 | |
| Rac-141 | 37 ± 2.7 | |
| | 236% | (relative to racemic) |
| | 61% | (relative to S) |

These results show that rac-141 has a substantially greater metabolic stability compared to pagoclone and to its (S) enantiomer ((S)-pagoclone).

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

What is claimed is:

1. A compound of Formula I 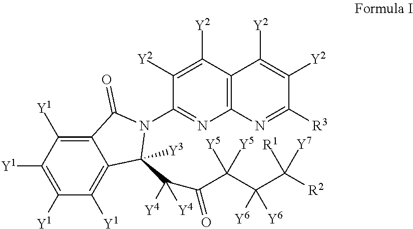
or a pharmaceutically acceptable salt thereof, wherein:
each Y¹ is the same and is hydrogen or deuterium;
each Y² is the same and is hydrogen or deuterium;
Y³ is hydrogen or deuterium;
each Y⁴ is the same and is hydrogen or deuterium;
each Y⁵ is the same and is hydrogen or deuterium;
each Y⁶ is the same and is hydrogen or deuterium;
Y⁷ is OR⁴, hydrogen or deuterium;
R¹ is CH₃ or CD₃;
R² is CH₃ or CD₃;
R³ is Cl, CH₃ or CD₃; and
R⁴ is hydrogen or P(O)(OR⁵)₂;
each R⁵ is independently hydrogen or C₁-C₆ alkyl, provided that at least one R⁵ is C₁-C₆ alkyl; provided that when each Y is hydrogen, at least one of R¹, R² and R³ is CD₃.

2. The compound of claim 1, wherein the compound has the Formula Ia 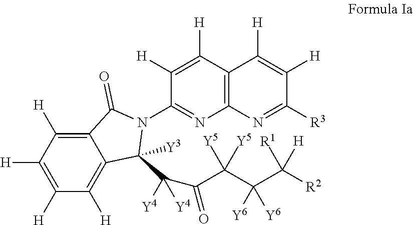
or a pharmaceutically acceptable salt thereof,
wherein:
Y³, Y⁴, Y⁵, Y⁶, R¹, R² and R³ are each defined as in Formula I, and wherein R¹ and R² are the same.

3. The compound of claim 1, wherein the compound has the Formula Ib 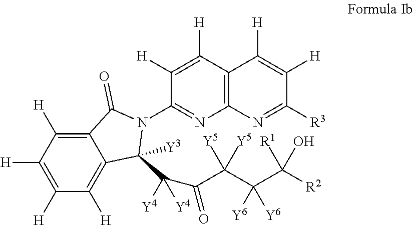
or a pharmaceutically acceptable salt thereof,
wherein:
Y³, Y⁴, Y⁵, Y⁶, R¹, R² and R³ are each defined as in Formula I, and wherein R¹ and R² are the same.

4. The compound of claim 1, wherein the compound has the Formula Ic 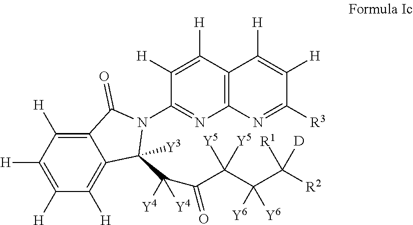
or a pharmaceutically acceptable salt thereof,
wherein:
Y³, Y⁴, Y⁵, Y⁶, R¹, R² and R³ are each defined as in Formula I, and wherein R¹ and R² are the same.

5. The compound of any of claim 1-4, wherein each Y⁶ is deuterium.

6. The compound of claim 5, wherein R¹ and R² are each CH₃.

7. The compound of claim 5, wherein R¹ and R² are each CD₃.

8. The compound of any of claims 1-4, wherein R³ is Cl.

9. The compound of any one of claims 1-4, wherein R³ is CD₃.

10. The compound of any one of claims 1-4, wherein each Y⁵ is hydrogen.

11. The compound of any one of claims 1-4, wherein each Y⁴ is hydrogen.

12. The compound of any one of claims 1-4, wherein R¹ and R² are each CD₃.

13. The compound of claim 12, wherein each Y⁶ is hydrogen.

14. The compound of claim 12, wherein R³ is Cl.

15. The compound of claim 12, wherein R³ is CD₃.

16. The compound of claim 12, wherein each Y⁵ is hydrogen.

17. The compound of claim 12, wherein each Y⁴ is hydrogen.

18. The compound of any one of claims 1-4, wherein any atom not designated as deuterium is present at its natural isotopic abundance.

19. The compound of claim 1, wherein Y³, each Y¹, each Y², each Y⁴ and each Y⁵ are all hydrogen, R¹ and R² are the same, and the compound is selected from any one of the compounds set forth in Table 1a below:
TABLE 1a
Examples of Compounds of Formula I

| Compound | R¹ = R² | R³ | each Y⁶ | Y⁷ |
|---|---|---|---|---|
| 101 | CD₃ | Cl | D | OH |
| 102 | CD₃ | Cl | D | H |
| 103 | CD₃ | Cl | H | OH |
| 104 | CD₃ | Cl | H | H |
| 105 | CD₃ | CD₃ | D | OH |
| 106 | CD₃ | CD₃ | D | H |
| 107 | CD₃ | CD₃ | H | OH |
| 108 | CD₃ | CD₃ | H | H |
| 109 | CH₃ | Cl | D | OH |
| 110 | CH₃ | Cl | D | H |
| 111 | CH₃ | Cl | H | OH |
| 112 | CH₃ | CD₃ | D | OH |
| 113 | CH₃ | CD₃ | D | H |
| 114 | CH₃ | CD₃ | H | OH |
| 115 | CH₃ | CD₃ | H | H |

or a pharmaceutically acceptable salt thereof, wherein any atom not designated as deuterium is present at its natural isotopic abundance.

20. The compound of claim 1, wherein Y³, each Y¹, each Y², each Y⁴ and each Y⁵ are all hydrogen, R¹ and R² are the same, and the compound is selected from any one of the compounds set forth in Table 1b below:
TABLE 1b
Examples of Compounds of Formula I

| Compound | R¹ = R² | R³ | each Y⁶ | Y⁷ |
|---|---|---|---|---|
| 121 | CD₃ | Cl | D | D |
| 122 | CD₃ | Cl | H | D |
| 123 | CD₃ | CD₃ | D | D |
| 124 | CD₃ | CD₃ | H | D |
| 125 | CH₃ | Cl | D | D |
| 126 | CH₃ | Cl | H | D |
| 127 | CH₃ | CD₃ | D | D |
| 128 | CH₃ | CD₃ | H | D |

or a pharmaceutically acceptable salt thereof, wherein any atom not designated as deuterium is present at its natural isotopic abundance

21. The compound of claim 1, wherein Y³, each Y¹, each Y², and each Y⁴ are all hydrogen, each Y⁵ is deuterium, R¹ and R² are the same, and the compound is selected from any one of the compounds set forth in Table 1c below:
TABLE 1c
Examples of Compounds of Formula I

| Compound | R¹ = R² | R³ | each Y⁶ | Y⁷ |
|---|---|---|---|---|
| 131 | CD₃ | Cl | D | D |
| 132 | CD₃ | Cl | H | D |
| 133 | CD₃ | CD₃ | D | D |
| 134 | CD₃ | CD₃ | H | D |
| 135 | CH₃ | Cl | D | D |
| 136 | CH₃ | Cl | H | D |
| 137 | CH₃ | CD₃ | D | D |
| 138 | CH₃ | CD₃ | H | D |

or a pharmaceutically acceptable salt thereof, wherein any atom not designated as deuterium is present at its natural isotopic abundance.

22. A pyrogen-free pharmaceutical composition comprising a compound of any of claims 1-4 or a pharmaceutically acceptable salt thereof; and a pharmaceutically acceptable carrier.

23. A method of treating in a subject a disease or condition selected from anxiety; agoraphobia; attention deficit hyperactivity disorder (ADHD); autism; bipolar disorder; dementia; insomnia; major depressive disorder; narcolepsy; obsessive-compulsive disorder (OCD); panic disorder; post-traumatic stress disorder (PTSD); schizophrenia; sleep disorder; social phobia; stuttering; Tourette's disorder; epilepsy; seizures; convulsions; neuropathic pain; inflammatory pain; migraine associated pain; and premature ejaculation, comprising administering to the subject a composition of claim 22.

24. The method of claim 23, wherein the disease or condition is general anxiety disorder; social anxiety; panic disorder; epilepsy; neuropathic pain, inflammatory pain; migraine associated pain; or premature ejaculation.

25. A compound of Formula A: 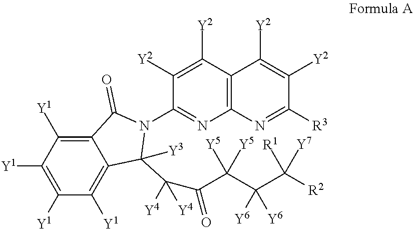
or a pharmaceutically acceptable salt thereof, wherein:
each Y¹ is the same and is hydrogen or deuterium;
each Y² is the same and is hydrogen or deuterium;
Y³ is hydrogen or deuterium;
each Y⁴ is the same and is hydrogen or deuterium;
each Y⁵ is the same and is hydrogen or deuterium;
each Y⁶ is the same and is hydrogen or deuterium;
Y⁷ is OR⁴, hydrogen or deuterium;
R¹ is CH₃ or CD₃;
R² is CH₃ or CD₃;
R³ is Cl, CH₃ or CD₃; and
R⁴ is hydrogen or P(O)(OR⁵)₂; wherein
each R⁵ is independently hydrogen or C₁-C₆ alkyl, provided that at least one R⁵ is C₁-C₆ alkyl;
provided that when each Y is hydrogen, at least one of R¹, R² and R³ is CD₃.

26. The compound of claim 25, wherein Y³, each Y¹, each Y², each Y⁴ and each Y⁵ are all hydrogen, R¹ and R² are the same, and the compound is a racemic mixture, wherein the compound is selected from any one of the compounds set forth in Table 3a below:
TABLE 3a
Examples of Compounds of Formula A

| Compound | R¹ = R² | R³ | each Y⁶ | Y⁷ |
|---|---|---|---|---|
| 301 | CD₃ | Cl | D | OH |
| 302 | CD₃ | Cl | D | H |
| 303 | CD₃ | Cl | H | OH |
| 304 | CD₃ | Cl | H | H |
| 305 | CD₃ | CD₃ | D | OH |
| 306 | CD₃ | CD₃ | D | H |
| 307 | CD₃ | CD₃ | H | OH |
| 308 | CD₃ | CD₃ | H | H |
| 309 | CH₃ | Cl | D | OH |
| 310 | CH₃ | Cl | D | H |
| 311 | CH₃ | Cl | H | OH |
| 312 | CH₃ | CD₃ | D | OH |
| 313 | CH₃ | CD₃ | D | H |
| 314 | CH₃ | CD₃ | H | OH |
| 315 | CH₃ | CD₃ | H | H |

or a pharmaceutically acceptable salt thereof, wherein any atom not designated as deuterium is present at its natural isotopic abundance.

27. The compound of claim 25, wherein Y³, each Y¹, each Y², each Y⁴ and each Y⁵ are all hydrogen, R¹ and R² are the same, the compound is a racemic mixture, and the compound is selected from any one of the compounds set forth in Table 3b below:
TABLE 3b
Examples of Compounds of Formula A

| Compound | R¹ = R² | R³ | each Y⁶ | Y⁷ |
|---|---|---|---|---|
| 321 | CD₃ | Cl | D | D |
| 322 | CD₃ | Cl | H | D |
| 323 | CD₃ | CD₃ | D | D |
| 324 | CD₃ | CD₃ | H | D |
| 325 | CH₃ | Cl | D | D |
| 326 | CH₃ | Cl | H | D |
| 327 | CH₃ | CD₃ | D | D |
| 328 | CH₃ | CD₃ | H | D |

or a pharmaceutically acceptable salt thereof, wherein any atom not designated as deuterium is present at its natural isotopic abundance.

28. The compound of claim 25, wherein Y³, each Y¹, each Y² and each Y⁴ are all hydrogen, each Y⁵ is deuterium, R¹ and R² are the same, the compound is a racemic mixture, and the compound is selected from any one of the compounds set forth in Table 3c below:
TABLE 3c
Examples of Compounds of Formula A

| Compound | R¹ = R² | R³ | each Y⁶ | Y⁷ |
|---|---|---|---|---|
| 331 | CD₃ | Cl | D | D |
| 332 | CD₃ | Cl | H | D |
| 333 | CD₃ | CD₃ | D | D |
| 334 | CD₃ | CD₃ | H | D |
| 335 | CH₃ | Cl | D | D |
| 336 | CH₃ | Cl | H | D |
| 337 | CH₃ | CD₃ | D | D |
| 338 | CH₃ | CD₃ | H | D |

or a pharmaceutically acceptable salt thereof, wherein any atom not designated as deuterium is present at its natural isotopic abundance.

29. The compound of claim 25, wherein Y³, each Y¹, and each Y² are all hydrogen, each Y⁴ and each Y⁵ are deuterium, R¹ and R² are the same, the compound is a racemic mixture, and the compound is selected from any one of the compounds set forth in Table 3d below:
TABLE 3d
Examples of Compounds of Formula A

| Compound | R¹ = R² | R³ | each Y⁶ | Y⁷ |
|---|---|---|---|---|
| 341 | CD₃ | Cl | D | D |
| 342 | CD₃ | Cl | H | D |
| 343 | CD₃ | CD₃ | D | D |
| 344 | CD₃ | CD₃ | H | D |
| 345 | CH₃ | Cl | D | D |
| 346 | CH₃ | Cl | H | D |
| 347 | CH₃ | CD₃ | D | D |
| 348 | CH₃ | CD₃ | H | D |
| 351 | CD₃ | Cl | D | OH |
| 352 | CD₃ | Cl | H | OH |
| 353 | CD₃ | CD₃ | D | OH |
| 354 | CD₃ | CD₃ | H | OH |
| 355 | CH₃ | Cl | D | OH |
| 356 | CH₃ | Cl | H | OH |
| 357 | CH₃ | CD₃ | D | OH |
| 358 | CH₃ | CD₃ | H | OH |
| 361 | CD₃ | Cl | D | H |
| 362 | CD₃ | Cl | H | H |
| 363 | CD₃ | CD₃ | D | H |
| 364 | CD₃ | CD₃ | H | H |
| 365 | CH₃ | Cl | D | H |
| 366 | CH₃ | Cl | H | H |
| 367 | CH₃ | CD₃ | D | H |
| 368 | CH₃ | CD₃ | H | H |

30. The compound of claim 1, wherein Y³, each Y¹, and each Y² are all hydrogen, each Y⁴ and each Y⁵ are deuterium, R¹ and R² are the same, and the compound is selected from any one of the compounds set forth in Table 1d below:
TABLE 1d
Examples of Compounds of Formula I

| Compound | R¹ = R² | R³ | each Y⁶ | Y⁷ |
|---|---|---|---|---|
| 141 | CD₃ | Cl | D | D |
| 142 | CD₃ | Cl | H | D |
| 143 | CD₃ | CD₃ | D | D |
| 144 | CD₃ | CD₃ | H | D |
| 145 | CH₃ | Cl | D | D |
| 146 | CH₃ | Cl | H | D |
| 147 | CH₃ | CD₃ | D | D |
| 148 | CH₃ | CD₃ | H | D |
| 151 | CD₃ | Cl | D | OH |
| 152 | CD₃ | Cl | H | OH |
| 153 | CD₃ | CD₃ | D | OH |
| 154 | CD₃ | CD₃ | H | OH |
| 155 | CH₃ | Cl | D | OH |
| 156 | CH₃ | Cl | H | OH |
| 157 | CH₃ | CD₃ | D | OH |
| 158 | CH₃ | CD₃ | H | OH |
| 161 | CD₃ | Cl | D | H |
| 162 | CD₃ | Cl | H | H |
| 163 | CD₃ | CD₃ | D | H |
| 164 | CD₃ | CD₃ | H | H |
| 165 | CH₃ | Cl | D | H |
| 166 | CH₃ | Cl | H | H |
| 167 | CH₃ | CD₃ | D | H |
| 168 | CH₃ | CD₃ | H | H |

or a pharmaceutically acceptable salt thereof, wherein any atom not designated as deuterium is present at its natural isotopic abundance.

31. A compound of Formula II 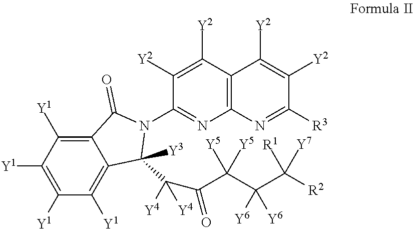
wherein Y¹, Y², Y³, Y⁴, Y⁵, Y⁶, Y⁷, R¹, R² and R³ are each defined as in Formula A in claim 25.

32. The compound of claim 31, wherein Y³, each Y¹, each Y² and each Y⁴ are all hydrogen, each Y⁵ is deuterium, R¹ and R² are the same, and the compound is selected from any one of the compounds set forth in Table 2c below:
TABLE 2c
Examples of Compounds of Formula II

| Compound | R¹ = R² | R³ | each Y⁶ | Y⁷ |
|---|---|---|---|---|
| 231 | CD₃ | Cl | D | D |
| 232 | CD₃ | Cl | H | D |
| 233 | CD₃ | CD₃ | D | D |
| 234 | CD₃ | CD₃ | H | D |
| 235 | CH₃ | Cl | D | D |
| 236 | CH₃ | Cl | H | D |
| 237 | CH₃ | CD₃ | D | D |
| 238 | CH₃ | CD₃ | H | D |

or a pharmaceutically acceptable salt thereof, wherein any atom not designated as deuterium is present at its natural isotopic abundance.

33. The compound of claim 31, wherein Y³, each Y^{I}, and each Y² are all hydrogen, each Y⁴ and each Y⁵ are deuterium, R¹ and R² are the same, and the compound is selected from any one of the compounds set forth in Table 2d below:
TABLE 2d
Examples of Compounds of Formula II

| Compound | R¹ = R² | R³ | each Y⁶ | Y⁷ |
|---|---|---|---|---|
| 241 | CD₃ | Cl | D | D |
| 242 | CD₃ | Cl | H | D |
| 243 | CD₃ | CD₃ | D | D |
| 244 | CD₃ | CD₃ | H | D |
| 245 | CH₃ | Cl | D | D |
| 246 | CH₃ | Cl | H | D |
| 247 | CH₃ | CD₃ | D | D |
| 248 | CH₃ | CD₃ | H | D |
| 251 | CD₃ | Cl | D | OH |
| 252 | CD₃ | Cl | H | OH |
| 253 | CD₃ | CD₃ | D | OH |
| 254 | CD₃ | CD₃ | H | OH |
| 255 | CH₃ | Cl | D | OH |
| 256 | CH₃ | Cl | H | OH |
| 257 | CH₃ | CD₃ | D | OH |
| 258 | CH₃ | CD₃ | H | OH |
| 261 | CD₃ | Cl | D | H |
| 262 | CD₃ | Cl | H | H |
| 263 | CD₃ | CD₃ | D | H |
| 264 | CD₃ | CD₃ | H | H |
| 265 | CH₃ | Cl | D | H |
| 266 | CH₃ | Cl | H | H |
| 267 | CH₃ | CD₃ | D | H |
| 268 | CH₃ | CD₃ | H | H |

or a pharmaceutically acceptable salt thereof, wherein any atom not designated as deuterium is present at its natural isotopic abundance.
